(19) United States
(12) Patent Application Publication
(10) Pub. No.: US 2021/0219953 A1
(43) Pub. Date: Jul. 22, 2021

(54) SYSTEMS FOR AN ULTRASOUND SCAN TRAY

(71) Applicant: GE Precision Healthcare LLC, Milwaukee, WI (US)

(72) Inventors: Ross Christopher Stalter, Hartland, WI (US); Doug Whisler, Burien, WA (US); Vijaykumar Viradia, Brookfield, WI (US); Sara Jane Good, Pleasanton, CA (US); Gabriela Romero, San Francisco, CA (US)

(21) Appl. No.: 16/747,203

(22) Filed: Jan. 20, 2020

(51) Int. Cl. A61B 8/00; A61B 8/08

(52) U.S. Cl. CPC A61B 8/4281; A61B 8/0825; A61B 8/4494

(57) ABSTRACT

Various methods and systems are provided for a scan tray of an ultrasound device. In one example, a system comprises a scan tray comprising a snap ring arrange between a base and a compressible material, wherein the snap ring comprises at least one hook configured to retain a membrane across the compressible material and onto the base.

### FIELD

Embodiments of the subject matter disclosed herein relate to an ultrasound scan tray with a removable acoustic membrane and patient comfort layer.

### BACKGROUND

Automated breast ultrasound screening (ABUS) may be used in conjunction with mammograms, to reveal abnormalities in dense breast tissue that may not be revealed in a mammogram. A field-of-view transducer is placed on a patient's chest and produces a 3-D image that can image through dense breast tissue without additional radiation. The ABUS may use a tray that is disposable so that a new tray is used for each patient to maintain hygiene requirements.

### BRIEF DESCRIPTION

In one embodiment, a system comprises a scan tray comprising a snap ring arranged between a base and a compressible material, wherein the snap ring comprises at least one hook configured to retain a membrane across the compressible material and onto the base.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1A shows a perspective view of a scanning apparatus;

FIG. 1B shows a schematic of various system components of a scanning apparatus;

FIG. 2 shows a first view of a scan tray;

FIG. 3 shows a second view of the scan tray, opposite the first view;

FIG. 4 shows a cross-section of the scan tray;

FIG. 5 shows the scan tray fitted to a scan head;

FIG. 6 shows a snap ring separated from a base of the scan tray; and

FIG. 7 shows a more detailed view of a membrane attached to the scan tray.

### DETAILED DESCRIPTION

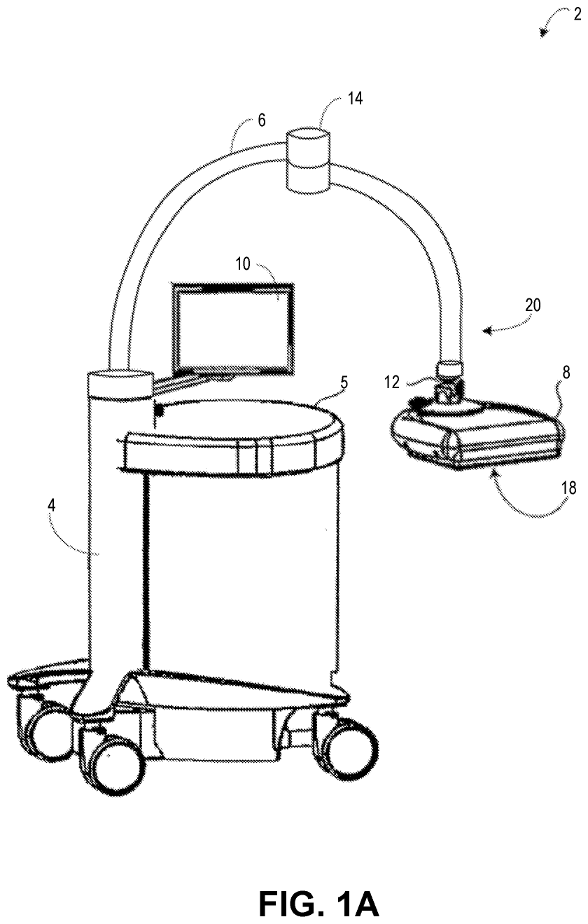

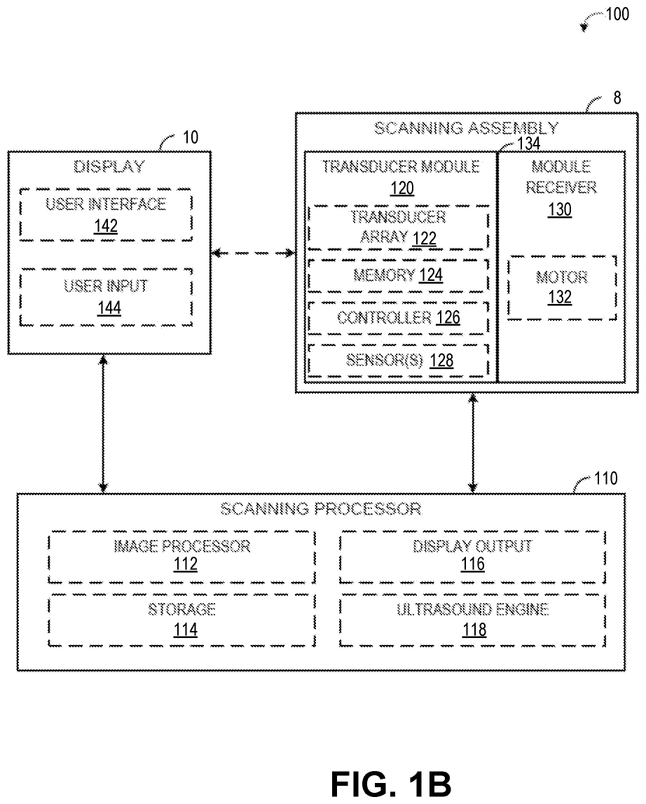

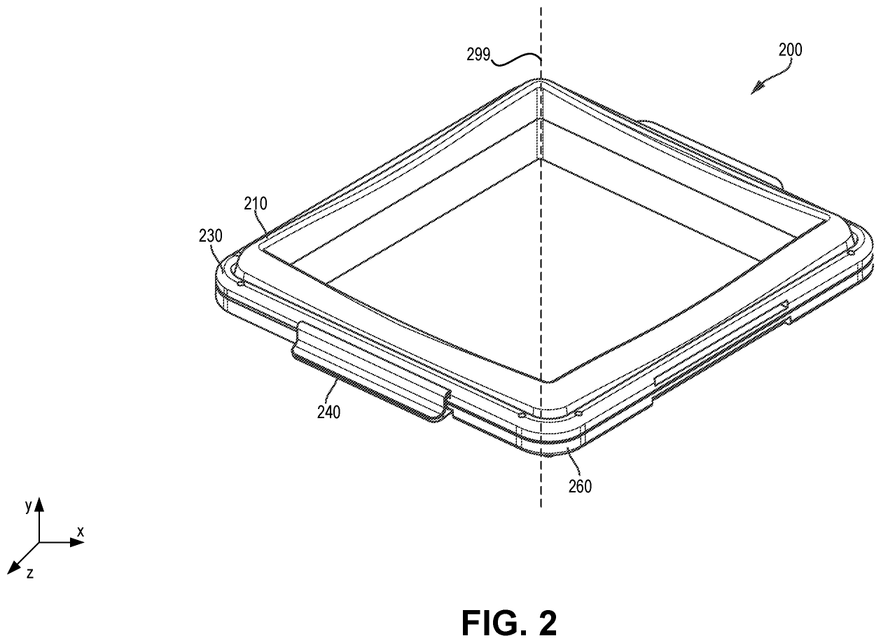

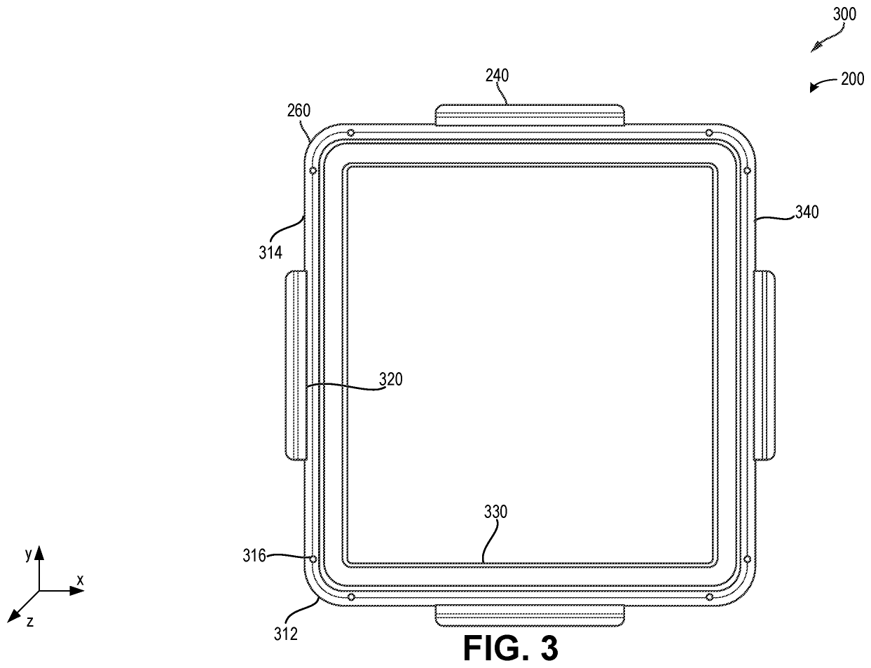

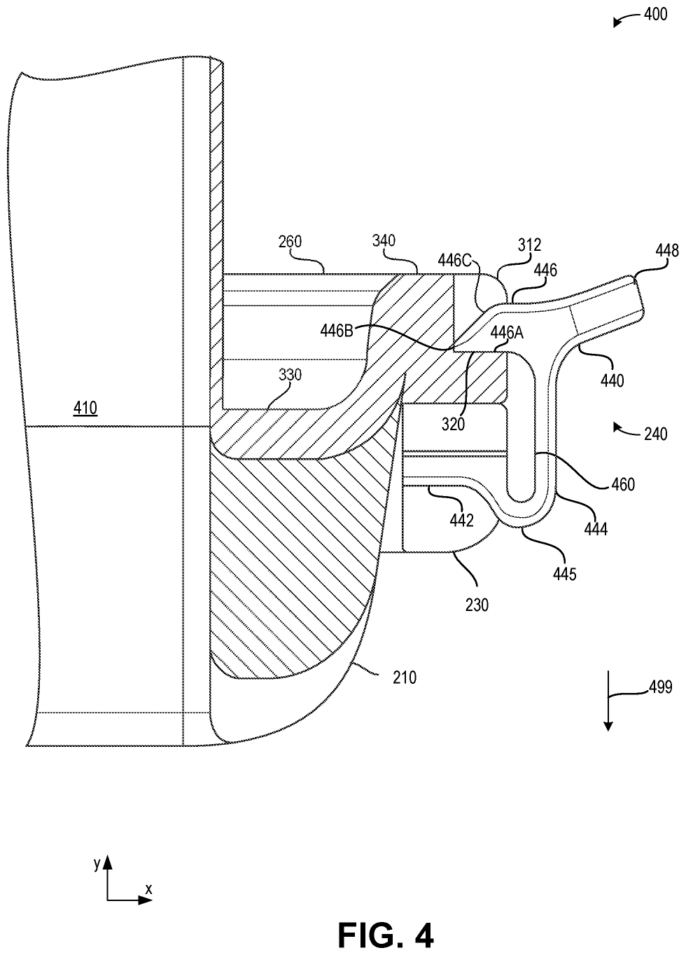

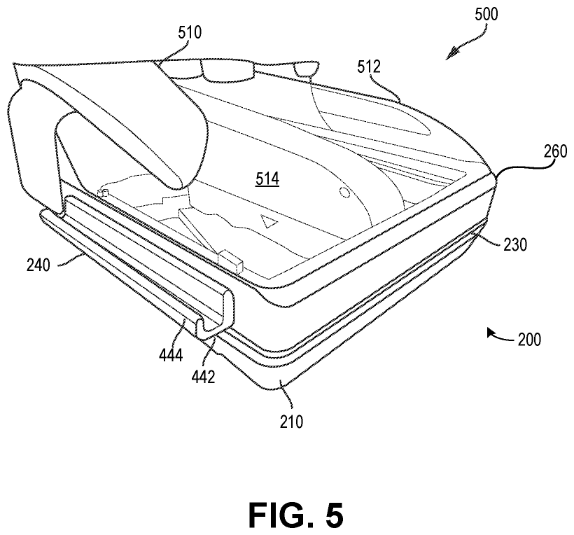

The following description relates to various embodiments of a scan tray attachment for a scan head. In one example, the scan tray is an attachment for a scan head of an ultrasound device for breast imaging. An example ultrasound system is illustrated in FIGS. 1A and 1B. First and second views of the scan tray are illustrated in FIGS. 2 and 3, respectively. The first view illustrates a first side of the scan tray that may interface with (e.g., contact) a patient and the second view illustrates a second side of the scan tray that may interface with the scan head. A cross-section of the scan tray is illustrated in FIG. 4, therein engagements (e.g., clips and/or hooks) are illustrated engaging with one or more cutouts (e.g., recesses) for lockingly securing the scan tray to the scan head. FIG. 5 illustrates the scan tray fitted to the scan head of an automated breast ultrasound screening device.

Before further discussion of the approach for a scan tray for an automated breast ultrasound screening device, some background discussion is provided. Previous examples of a scan tray included a membrane, such as an acoustic membrane, heat-staked to a frame of the scan tray. After completing imaging for a first patient, the entire unit (e.g., the scan tray with the membrane) may be disposed by an operator and the ultrasound device was prepared for imaging with a second patient, resulting in relatively high waste and costs.

In one example, a scan tray comprises a removable acoustic membrane coupled to an at least two-part frame. The acoustic membrane may be disposed after a single use while the frame and remaining portion of the scan tray may be reused. The acoustic membrane may comprise one or more types of material including but not limited to chiffon, civflex, or a combination of one or more membranes. The ability to reuse the frame and to be able to use a hydrophilic and impenetrable membrane material may save money, reduce waste, and reduce cleaning operations. The reusable frame may also comprise a third part, which includes a compressible material that increases patient comfort by softening an area of the frame that contacts the patient. Thus, in one example, the issues of the previous example are at least partially solved by a reusable scan tray configured to decrease consumption of scan gels and/or scan lotions and an acoustic membrane that is impenetrable, thereby blocking the scan fluids that contact the patient from entering the scan head or contacting the transducer. This decreases cleaning time and the spread of contaminants from patient to patient.

In one aspect, a scan tray may be configured to engage with a scan head of an ultrasound device. The scan tray may comprise a locking feature and a comfort feature to both enhance patient comfort and decrease operator clean up times. The technical effect of integrating the locking feature and the comfort feature into the scan tray is to decrease clean-up times and enhance patient experience.

X-ray mammography is the most commonly used imaging method for mass breast cancer screening. However, x-ray mammograms only detect a summation of the x-ray opacity of individual slices over the entire breast. Alternatively, ultrasound imaging can separately detect sonographic properties of individual slices of breast tissue, thereby enabling users to detect breast lesions where x-ray mammography alone may fail.

In one example, volumetric ultrasound scanning of the breast may be used as a complementary modality for breast cancer screening. Volumetric ultrasound scanning may include moving an ultrasound transducer relative to a tissue sample and then processing the resultant ultrasound echoes to form a data volume representing at least one acoustic property of the tissue sample. Another well-known shortcoming of x-ray mammography practice is found in the case of dense-breasted women, including patients with high content of fibroglandular tissues in their breasts. Because fibroglandular tissues have higher x-ray absorption than the surrounding fatty tissues, portions of breasts with high fibroglandular tissue content are not well penetrated by x-rays and thus the resulting mammograms contain reduced information in areas where fibroglandular tissues reside. Thus, the use of volumetric ultrasound scanning in conjunction with conventional x-ray mammography may increase the early breast cancer detection rate.

In one example, a full-field breast ultrasound (FFBU) scanning apparatus, such as the FFBU scanning apparatus depicted in FIGS. 1A and 1B, compresses a breast in a generally chestward or head-on direction and ultrasonically scans the breast. In another example, the FFBU scanning apparatus may compress a breast along planes such as the craniocaudal (CC) plane, the mediolateral oblique (MLO) plane, or the like. A compression/scanning assembly of the FFBU scanning apparatus may include an at least partially conformable, substantially taut membrane or film sheet, an ultrasound transducer, and a transducer translation mechanism. One side of the taut membrane or film sheet compresses the breast. The transducer translation mechanism maintains the ultrasound transducer in contact with the other side of the film sheet while translating the ultrasound transducer thereacross to scan the breast.

Although several examples herein are presented in the particular context of human breast ultrasound, it is to be appreciated that the present teachings are broadly applicable for facilitating ultrasonic scanning of any externally accessible human or animal body part (e.g., abdomen, legs, feet, arms, neck, etc.). Moreover, although several examples herein are presented in the particular context of mechanized scanning (i.e., in which the ultrasound transducer is moved by a robot arm or other automated or semi-automated mechanism), it is to be appreciated that one or more aspects of the present teachings can be advantageously applied in a handheld scanning context.

FIG. 1A illustrates a perspective view of a full-field breast ultrasound (FFBU) scanning apparatus 2 according to an embodiment, comprising a frame 4 that contains an ultrasound processor, a movable and adjustable support arm 6 (e.g., adjustable arm) including a hinge joint 14, a compression/scanning assembly 8 connected to the adjustable arm 6 via a ball-and-socket connector (e.g., ball joint) 12, and a display 10 connected to the frame 4. The display 10 is coupled to the frame 4 at an interface where the adjustable arm 6 enters into the frame 4. As a result of being directly coupled to the frame 4 and not to the adjustable arm 6, the display 10 does not affect a weight of the adjustable arm 6 and a counterbalance mechanism of the adjustable arm 6. In one example, the display 10 is rotatable in a horizontal and lateral direction (e.g., rotatable around a central axis of the frame 4), but not vertically movable. In an alternate example, the display 10 may also be vertically movable. While FIG. 1A depicts the display 10 coupled to the frame 4, in other examples the display 10 may be coupled to a different component of the scanning apparatus 2, such as coupled to the ultrasound processor housing 105, or located remotely from the scanning apparatus 2.

In one embodiment, the adjustable arm 6 is configured and adapted such that the compression/scanning assembly 108 is either (i) neutrally buoyant in space, or (ii) has a light net downward weight (e.g., 1-2 kg) for breast compression, while allowing for easy user manipulation. In alternate embodiments, the adjustable arm 6 is configured such that the compression/scanning assembly 1088 is neutrally buoyant in space during positioning the scanner on the patient's tissue. Then, after positioning the compression/scanning assembly 8, internal components of the adjustable arm 6 may be adjusted to apply a desired downward weight for breast compression and increased image quality. In one example, the downward weight (e.g., force) may be in a range of 2-11 kg.

As introduced above, the adjustable arm 6 includes a hinge joint 14. The hinge joint 14 bisects the adjustable arm 6 into a first arm portion and a second arm portion. The first arm portion is coupled to the compression/scanning assembly 8 and the second arm portion is coupled to the frame 4. The hinge joint 14 allows the second arm portion to rotate relative to the second arm portion and the frame 4. For example, the hinge joint 14 allows the compression/scanning assembly 8 to translate laterally and horizontally, but not vertically, with respect to the second arm portion and the frame 4. In this way, the compression/scanning assembly 8 may rotate toward or away from the frame 4. However, the hinge joint 14 is configured to allow the entire adjustable arm 6 (e.g., the first arm portion and the second arm portion) to move vertically together as one piece (e.g., translate upwards and downwards with the frame 4).

The compression/scanning assembly 8 comprises an at least partially conformable membrane 18 in a substantially taut state for compressing a breast, the membrane 18 having a bottom surface contacting the breast while a transducer is swept across a top surface thereof to scan the breast. In one example, the membrane is a taut fabric sheet. Optionally, the adjustable arm 6 may comprise potentiometers (not shown) to allow position and orientation sensing for the compression/scanning assembly 8, or other types of position and orientation sensing (e.g., gyroscopic, magnetic, optical, radio frequency (RF)) can be used. Within frame 4 may be provided a fully functional ultrasound engine for driving an ultrasound transducer and generating volumetric breast ultrasound data from the scans in conjunction with the associated position and orientation information. The volumetric scan data can be transferred to another computer system for further processing using any of a variety of data transfer methods known in the art. A general purpose computer, which can be implemented on the same computer as the ultrasound engine, is also provided for general user interfacing and system control. The general purpose computer can be a self-contained stand-alone unit, or can be remotely controlled, configured, and/or monitored by a remote station connected across a network.

As will be described in greater detail herein, the compression/scanning assembly 8 may be divided into two parts, including a scan head and a scan tray. The membrane 18 may be coupled to the scan tray, which may be optionally coupled to the scan head. An advantage of dividing the compression/scanning assembly 8 into the scan head and the scan tray is to decrease clean up times between patients as the scan tray of the present disclosure may enable quicker disposal of the membrane 18 with reduced cleaning of the scan tray and/or scan head. The scan tray further comprises features which engage the membrane 18 and tighten it such that the membrane 18 is taut and configured to compress a breast or other body part.

FIG. 1B is a block diagram 100 schematically illustrating various system components of the scanning apparatus 2, including the scanning assembly 8, display 10, and a scanning processor 110. Scanning processor 110 may be included within frame 4 of the scanning apparatus 2 in one example. As illustrated in the embodiment of FIG. 1B, the scanning assembly 8, display 10, and scanning processor 110 are separate components in communication with each other; however, in some embodiments one or more of the components may be integrated (e.g., the display and scanning processor may be included in a single component).

Referring first to the scanning assembly 8, it comprises a transducer module 120 connected to a module receiver 130. The module receiver 130 may be positioned within a housing (attached to the arm 6 of the scanning apparatus 2 of FIG. 1, for example) that is configured to remain stationary during scanning, while the module receiver 130 is configured to translate with respect to the housing during scanning. In order to automatically translate with respect to the housing during scanning, the module receiver includes a motor 132 activated by the scanning processor 110, as explained below.

The transducer module 120 comprises a transducer array 122 of transducer elements, such as piezoelectric elements, that convert electrical energy into ultrasound waves and then detect the reflected ultrasound waves. The transducer module 120 is configured to be removably coupled with the module receiver 130 via a connection 134. The connection 134 may include complementary connectors on the transducer module and module receiver (e.g., a first connector on the transducer module that is configured to connect with a second connector on the module receiver) in order to establish both a mechanical connection and an electrical connection between the module receiver and the transducer module.

The transducer module 120 may further include a memory 120. Memory 124 may be a non-transitory memory configured to store various parameters of the transducer module 120, such as transducer usage data (e.g., number of scans performed, total amount of time spent scanning, etc.), as well as specification data of the transducer (e.g., number of transducer array elements, array geometry, etc.) and/or identifying information of the transducer module 120, such as a serial number of the transducer module. Memory 124 may include removable and/or permanent devices, and may include optical memory, semiconductor memory, and/or magnetic memory, among others. Memory 124 may include volatile, nonvolatile, dynamic, static, read/write, read-only, random-access, sequential-access, and/or additional memory. In an example, memory 124 may include RAM. Additionally or alternatively, memory 124 may include EEPROM.

Memory 124 may store non-transitory instructions executable by a controller or processor, such as controller 126, to carry out one or more methods or routines as described herein below. Controller 126 may receive output from various sensors 128 of the transducer module 120 and trigger actuation of one or more actuators and/or communicate with one or more components in response to the sensor output. Sensors 128 may include one or more pressure sensors and/or one or more temperature sensors. During scanning, the pressure across the scanning assembly 8 may be measured by the pressure sensors, and if the pressure distribution across the transducer module is not equal, a user may be notified (via user interface 142 of display 10, for example) to reposition the scanning assembly 8. Further, in some embodiments, to initiate scanning, motor 132 may be activated via a signal from controller 126. However, in other embodiments, motor 132 may be activated via a signal from a separate scanning processor 110, explained below.

Scanning assembly 8 may be in communication with scanning processor 110, to send raw scanning data to an image processor, for example. Additionally, data stored in memory 124 and/or output from sensors 128 may be sent to scanning processor 110 in some examples. Further, various actions of the scanning assembly 108 (e.g., translation of the module receiver 130, activation of the transducer elements, etc.) may be initiated in response to signals from the scanning processor 110. Scanning assembly 8 may optionally communicate with display 10, in order to notify a user to reposition the scanning assembly, as explained above, or to receive information from a user (via user input 144), for example.

Turning now to scanning processor 110, it includes an image processor 112, storage 114, display output 116, and ultrasound engine 118. Ultrasound engine 118 may drive activation of the transducer elements of the transducer array 122 of transducer module 120 and, in some embodiments, may activate motor 132. Further, ultrasound engine 118 may receive raw image data (e.g., ultrasound echoes) from the scanning assembly 8. The raw image data may be sent to image processor 112 and/or to a remote processor (via a network, for example) and processed to form a displayable image of the tissue sample. It is to be understood that the image processor 112 may be included with the ultrasound engine 118 in some embodiments.

Information may be communicated from the ultrasound engine 118 and/or image processor 112 to a user of the scanning apparatus 2 via the display output 116 of the scanning processor 110. In one example, the user of the scanning apparatus may include an ultrasound technician, nurse, or physician such as a radiologist. For example, processed images of the scanned tissue may be sent to the display 10 via the display output 116. In another example, information relating to parameters of the scan, such as the progress of the scan, may be sent to the display 10 via the display output 116. The display 10 may include a user interface 142 configured to display images or other information to a user. Further, user interface 142 may be configured to receive input from a user (such as through user input 144) and send the input to the scanning processor 110. User input 144 may be a touch screen of the display 10 in one example. However, other types of user input mechanisms are possible, such as a mouse, keyboard, etc.

Scanning processor 110 may further include storage 114. Similar to memory 124, storage 114 may include removable and/or permanent devices, and may include optical memory, semiconductor memory, and/or magnetic memory, among others. Storage 114 may include volatile, nonvolatile, dynamic, static, read/write, read-only, random-access, sequential-access, and/or additional memory. Storage 114 may store non-transitory instructions executable by a controller or processor, such as ultrasound engine 118 or image processor 112, to carry out one or more methods or routines as described herein below. Storage 114 may store raw image data received from the scanning assembly 8, processed image data received from image processor 112 or a remote processor, and/or additional information.

Turning now to FIG. 2, it shows a first view of a scan tray 200. The scan tray 200 may be configured to mate with a scan head, which may be a portion of the compression/scanning assembly 8 of the scanning apparatus 2 of FIGS. 1A and 1B. The example of FIG. 2 further comprises an axis system comprising three axes, namely an x-axis parallel to a horizontal direction, a y-axis parallel to a vertical position, and a z-axis perpendicular to each of the x- and y-axes. A central axis 299 extends through a geometric center of the scan tray 200 in a direction parallel to the y-axis.

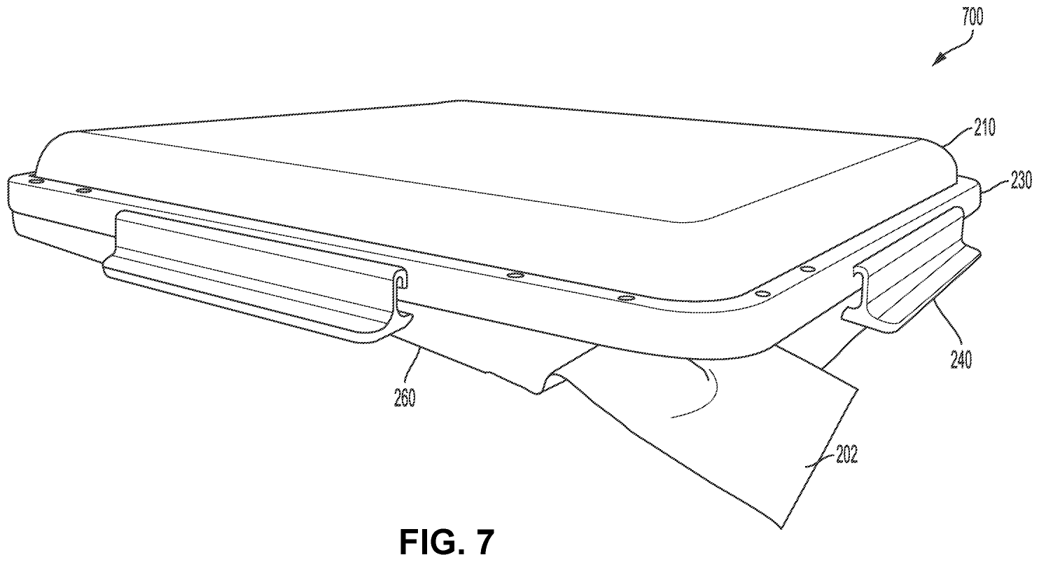

The scan tray 200 comprises a compressible material 210, a snap ring 230, and a base 260. The snap ring 230 may further comprise at least one hook 240. A membrane 202, which is omitted in the example of FIG. 2 and illustrated in the example of FIG. 7, may be an acoustic membrane in one example, may be arranged over the compressible material 210 and maintained in a taut position via the at least one hook 240 pressing the membrane 202 into engaging features of the base 260. In one example, the engaging is a pinching. The membrane 202 may be a non-limiting example of membrane 18 of FIG. 1A.

Each of the compressible material 210, the snap ring 230, and the base 260 may comprise a generally square-shaped cross-section taken along the x-z plane. The snap ring 230 may be positioned between the compressible material 210 and the base 260.

The base 260 may be configured to mate with an existing shape of a scan head. In one example, the base 260 may be free of ribs and other protruding features to enable a tight fit between the base 260 and the scan head. Additionally or alternatively, the base 260 may be free of ribs and the like to accommodate one or more magnets of the scan head and/or the scan tray 200. In one example, the base 260 or another portion of the scan tray 200 may be magnetic and attracted to magnets of the scan head, thereby holding the scan tray against the scan head. The base 260 is described in greater detail below.

The compressible material 210 may be configured to interface with a patient. In one example, the compressible material 210 may be sized such that a breast or other body part proximal to the patient's chest may be received within an opening of the compressible material 210. The compressible material 210 may contact one or more of a clavicle, a sternum, one or more ribs, and a region nearby the preceding areas of the patient. The compressible material 210 may be configured to reduce an amount of force applied to the patient when imaging is occurring. In one example, the compressible material 210 is a foam or other compressible material which may allow a closer imaging proximity between the scan head and the patient along with improved patient comfort.

The compressible material 210 may further provide at least some force against the membrane 202, wherein the force is similar to a spring force in one example. The force, in combination with the at least one hook 240, may block the membrane 202 from loosening, which may result in reduced image quality. Furthermore, if the membrane 202 is loose, scan fluids may leak around the membrane and contact portions of the scan tray 200, resulting in longer clean-up times.

In some examples, the compressible material 210 may be omitted from the scan tray 200 without departing from the scope of the present disclosure.

Examples of the compressible material 210 may include open or closed-cell foams. In one example, the compressible material 210 is a foam rubber. In another example, the compressible material 210 is a polyurethane foam or a silicone foam. A compressibility of the compressible material 210 may be greater than or equal to 50% of a thickness of the compressible material 210. As such, the compressible material 210 may be reduced in thickness as the compressible material 210 is pressed against a surface of the patient while the snap ring 230 and the base 260 may remain fully expanded with minimal to zero compression.

The membrane 202 may comprise a plurality of materials. In one example, the membrane 202 comprises chiffon, which is a light, sheer fabric, and is an acoustic membrane. The membrane 202 may be sheer (e.g., transparent) to assist an operator with desirably positioning a scan head.

The membrane 202 may be replaceable and configured to be used individually, with a replicate of itself, or with a materially different membrane. The membrane may be hydrophilic, which may allow the membrane 202 to self-lubricate, thereby decreasing a scan gel and/or scan lotion demand. Furthermore, the membrane 202 and the method of retaining the membrane 202 via the snap ring 230 may block fluids from entering the scan tray, the scan head, and/or a transducer, thereby limiting clean up times and transfer from patient to patient. In one example, the membrane 202 extends over an outer surface of the compressible material 210 and is secured to recesses or the base via at least one hook 240. As such, the membrane 202 is arranged between the patient and the compressible material 210 during an imaging procedure.

The snap ring 230 comprises at least one hook 240. The at least one hook 240 may be configured to engage with (e.g., lock with) a recess of the base 260. In one example, the membrane 202 may wrap and engage in face-sharing contact with a locking portion of the hook 240 such that the membrane 202 is pressed against the recess of the base 260 via the at least one hook 240. This may increase a tautness of the membrane 202, which may enhance an image quality. As such, a combination of the spring force of the compressible material 210 and the locking action of the snap ring 230 via the at least one hook 240 may decrease a looseness of the membrane 202.

The at least one hook 240 may be one of a plurality of hooks. Each hook may be arranged on a different side of the snap ring 230. In the example of FIG. 2, the snap ring 230 comprises four sides. One hook may be arranged on each of the sides of the snap ring 230. As such, the hooks may be symmetrically arranged about the snap ring 230 to evenly distribute a force against the membrane 202. Herein, the at least one hook 240 is referred to as a plurality of hooks 240.

In one example, the snap ring 230 and the base 260 comprises substantially similar widths, measured along an x-z plane. The compressible material 210 may comprise a width less than the widths of the snap ring 230 and the base 260. In one example, such as the example illustrated in FIG. 2, the compressible material 210 is physically coupled directly to an inner width of the base 260 and the snap ring 230 is physically coupled directly to an outer width of the base 260. The compressible material 210 may curve slightly inward toward an opening between interior walls of the snap ring 230 and the base 260 at a center of each segment of the compressible material 210. The snap ring 230 and the compressible material 210 may be in face-sharing contact at an interface between the snap ring 230 and the compressible material 210 with the base 260. The plurality of hooks 240 extend beyond a profile of the outer width of the base 260. The plurality of hooks 240 may be configured to articulate at some angle to receive the membrane 202 and then carry the membrane 202 to an engagement feature (e.g., the recess) of the base 260.

In one example, each of the plurality of hooks 240 are configured to articulate at least 90 degrees away from the central axis of the scan tray (e.g., central axis 299 of FIG. 2). In some examples, the plurality of hooks 240 are configured to actuate between 15 to 180 degrees. In some examples, additionally or alternatively, the plurality of hooks 240 are configured to actuate between 50 to 160 degrees. In some examples, additionally or alternatively, the plurality of hooks 240 are configured to actuate between 80 and 135 degrees. In one example, the plurality of hooks 240 are configured to actuate between 90 and 120 degrees.

The scan tray 200, including the base 260, the snap ring 230, and the compressible material may be constructed via additive manufacturing (e.g., 3-D printing). The membrane 202, which may comprise chiffon and/or civflex, may be retained onto the base 260 via the hooks 240. As such, the membrane 202 is releasably retained onto the base 260 via the snap ring 230.

Turning now to FIG. 3, it shows a second view 300 of the scan tray 200. In the second view 300, the compressible material 210 is omitted from view via the base 260 and the snap ring 230. In the example of FIG. 3, the plurality of hooks 240 are shown in an engaged position, such that the snap ring 230 and/or a membrane are irremovable from the base 260. The engaged position may be interchangeably referred to as a locking position, herein.

The base 260 comprises rounded corners 312 arranged between each side of a plurality of sides 314 of the base 260. In one example, the base 260 comprises a shape substantially similar to a shape of the snap ring 230, wherein each of the base 260 and the snap ring 230 comprises a substantially square shape with contoured (e.g., curved) corners. The base 260 further comprises a plurality of through-holes 316 through which a fastener may be extended. The fastener may physically couple the base 260 to the snap ring 230. In one example, two through-holes of the plurality of through-holes 316 may be spaced about each corner of the corners 312. Said another way, a side of the plurality of sides 314 comprises two through-holes, wherein the through-holes of a single side are arranged distal to one another and proximal to respective corners associated with the side.

Each side of the plurality of sides 314 further comprises at least one engagement feature 320, wherein the engagement feature is configured to receive a portion of a hook of the plurality of hooks 240. In one example, such as shown in the example of FIG. 3, the engagement feature receives an arm or other similar component of the hook and blocks its release without operator input. A detailed view of a single engagement feature 320 engaging with a single hook is illustrated in FIG. 4. In one example, the engagement feature 320 is a recess 320.

The base 260 further comprises an interior depression 330. The interior depression 330 may be arranged directly opposite a portion of the base 260 configured to receive the compressible material (e.g., compressible material 210 of FIG. 2). The interior depression 330 may be arranged on an inner width of the base 260 relative to an outer body 340, wherein the outer body 340 comprises the through-holes 316 and the at least one engagement features 320. The interior depression 330 may be shaped to receive a frame of the scan head of the ultrasound device. In one example, the scan head is force fit into the interior depression 330. Additionally or alternatively, the interior depression 330 may comprise one or more coupling features, such as magnets, interlocking pins and tabs, and/or other coupling features. When the scan head is inserted into the interior depression 330, the outer body 340 may occlude a portion of the scan head. Said another way, the outer body 340 may at least partially surround the scan head when the scan head is positioned into the interior depression 330.

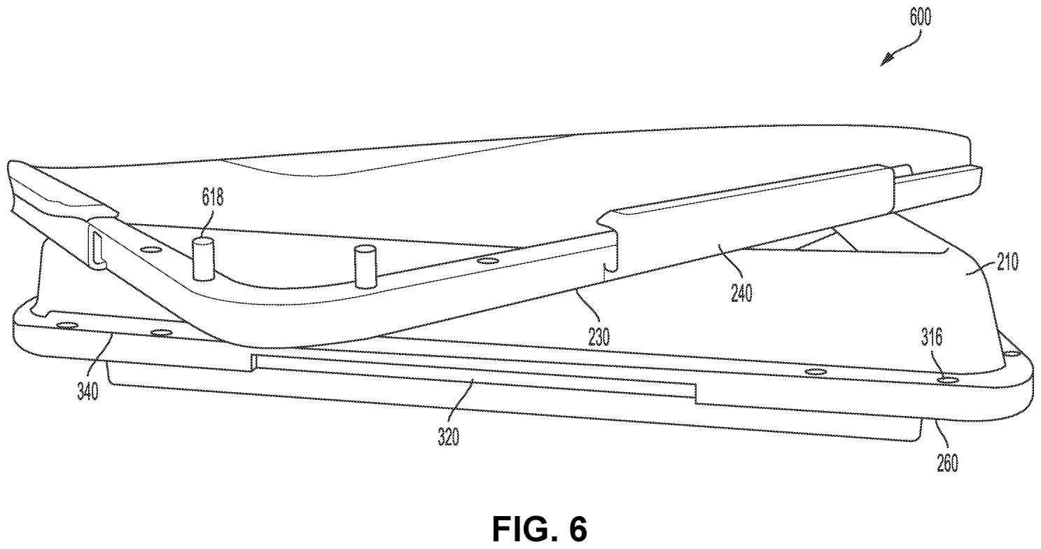

Turning to FIG. 6, it shows an embodiment 600 of the snap ring 230 separated from the base 260. The through-holes 316 and a plurality of corresponding fasteners 318 are illustrated in greater detail. As illustrated, the compressible material 210 is fixedly coupled to the base 260 independent of the snap ring 230. The plurality of fasteners 618 comprise a cylindrical shape configured to insert into the through-holes 316. In one example, the fasteners 618 are dowels. In another example, the fasteners 618 are pins. Additionally or alternatively, the fasteners 618 may comprise magnets and other engaging features (e.g., click-lock). Each fastener of the fasteners 318 may be inserted into a single corresponding through-hole of the through-holes 316.

Turning to FIG. 7, it shows an embodiment 700 of the snap ring 230 physically coupled to the base 260. Additionally, the embodiment 700 comprises the membrane 202 arranged over the compressible material 210. As illustrated, the membrane 202 extend over the compressible material 210 and over the outer body (e.g., outer body 340 of FIG. 3) of the base 260. Following arranging the membrane 202 over the compressible material 210 and the outer body of the base 260, the snap ring 230 may be aligned with the base 260 so that the plurality of fasteners is inserted into a plurality of corresponding through-holes (e.g., fasteners 618 and through-holes 216 of FIG. 6). In one example, the plurality of fasteners may press portions of the membrane 202 into the through-holes 216, which may increase a tautness of the membrane 202. Once the membrane is aligned and its fasteners are inserted into the through-holes, the hooks 240 may be actuated, wherein the hooks 240 are configured to push a portion of the membrane 202 into corresponding engagement features (e.g., recesses). In one example, the hooks 240 pinch the membrane 202 into the recess and block the membrane from separating from the scan tray until the hooks 240 are released. By doing this, the membrane 202 may be fully retained and taut, which may allow the membrane 202 to compress a breast during an imaging procedure. Said another way, when the membrane is fully retained to the scan tray and taut, the membrane 202 may block the snap ring 230 from directly contacting the base 260 and the compressible material 210 due to the membrane 202 being sandwiched between the snap ring and the base and the compressible material.

In one example, the snap ring 230 may function as a collar or other similar device that press fits the membrane 202 into the base 260 via the fasteners 618. The snap ring 230 then further couples the membrane 202 to the base 260 via the hooks 240 which may pinch portions of the membrane 202 into the recesses of the base 260.

In one example, a method for an ABUS imaging procedure may include physically coupling the base to a frame of a scan head. The method further includes arranging a membrane over a surface of the scan tray facing away from the scan head, wherein the membrane is in face-sharing contact with a compressible material and the base. The method further comprises aligning fasteners of a snap ring with through-holes of the base and pressing the snap ring against the base to insert the fasteners through corresponding alignment holes. As such, the membrane may also be pushed into the through-holes via the fasteners. The method further comprises pressing hooks into the recesses of the base, wherein the hooks pinch portions of the membrane into the recesses. The membrane may be blocked from escaping from the recesses until an operator actuates the hooks away from the recesses. As such, the membrane may be taut and the scan head and scan tray configured to perform a scanning procedure. Upon completing a scanning procedure, an operator may unlatch the hooks by applying a force against the hook away from the recesses. By doing this, the membrane may no longer be pinched into the recesses. The operator may then move the entire snap ring away from the base, which may remove the fasteners from the through-holes and completely release the membrane from the base and the compressible material. The membrane may be disposed and replaced with a new, unused membrane for a future scanning procedure, if desired.

Turning now to FIG. 4, it shows a cross-section 400 of one recess 320 engaging with one hook 440 of the plurality of hooks 240. In the position illustrated in the example of FIG. 4, the hook 440 is in a locked position.

The hook 440 generally comprises a J-shape. The hook 440 comprises a reduced thickness portion 442 coupled to the snap ring 230. The reduced thickness portion 442 may comprise a reduced thickness relative to the thickness of the snap ring 230. A bend 444 extends from the reduced thickness portion 442 toward an arm 446 and a tab 448 of the hook 440. The bend 444 may generally comprise a U-shape wherein first and second extreme ends of the bend 444 point in similar direction. However, a body of the bend 444 may curve around an extension 460 of the outer body 340 of the base 260. The extension 460 may comprise an L-shape, wherein a first portion of the L-shape may correspond to the recess 320 and a second portion may be surrounded by the bend 444.

The bend 444 may be flexible at its curve 445, arranged between the first and second extreme ends of the bend 444. In one example, the curve 445 is biased toward the first end adjacent to the reduced thickness portion 442. As such, the bend 444 may flex and/or actuate at the curve 445 such that the hook 440 may move about the extension 460.

The arm 446 extends in a first direction from the bend 444 and the tab 448 extends in a second direction, opposite the first direction. In one example, the first direction is toward the scan tray 200 and the second direction is away from the scan tray 200. The arm 446 comprises a cross-section having a generally triangular shape. That is to say, the arm 446 comprises a first surface 446A which is in face-sharing contact with the recess 320 and a second surface 446B, which is smaller than the first surface 446A and in face-sharing contact with the outer body 340. The arm 446 further comprises a third surface 446C which is beveled and angled to each of the outer body 340 and the recess 320. In one example, the second surface 446B is smaller than the first surface 446A, which is equal in size to the third surface 446C.

The hook 440 may be contoured as it transitions from the third surface 446C to the tab 448. The tab 448 may comprise a substantially rectangular shape. The tab 448 may be configured to release the arm 446 from the recess 320 in response to a force in the direction 499. In one example, the direction 499 is parallel to gravity. Furthermore, the force may exceed a threshold force in order to decouple the arm 446 from the recess 320. In one example, the threshold force may be based on a force sufficient to block the decoupling due to inadvertent contact with the tab 448 while still allowing the operator to quickly and easily decouple the arm 446 from the recess 320 to remove the membrane for a subsequent patient scan.

Distal to the locking features, there is an interior passage 410 through which sound waves of the ultrasound may pass for imaging. The interior passage 410 may be shaped by both the base 260 and the compressible material 210. The membrane 202 covers an outlet of the interior passage 410 at the compressible material 210.

In one example, the interior passage 410 is an opening and/or an expanse, wherein ultrasound pulses may travel freely. The interior passage 410 may be shaped via interior edges of a combination of the base, the compressible material, and the snap ring. In one example, only the base and the compressible material directly shape the interior passage 410. The membrane 202 may extend across an entirety of the interior passage 410. More specifically, the membrane 202 extends across an extreme end of the interior passage 410 sound pulses from the interior passage 410 may reach a patient or other desired object. In one example, the interior passage 410 is continuous and uninterrupted, wherein none of the base 260, the compressible material 210, and the snap ring 230 extend into an area of the interior passage 410. Furthermore, the membrane 202 is continuous and uninterrupted, and wherein the membrane is coupled to the base 260 at regions of the base 260 away from the interior passage 410.

Turning now to FIG. 5, it shows an example ultrasound system 500 comprising the scan tray 200. The ultrasound system 500 comprises an articulating arm 510 coupled to a scan head 512 comprising a transducer 514. The base 260 may interface with the scan head 512 and lock thereto so that the base 260 is physically coupled to the scan head 512. As the articulating arm 510 is actuated to position the scan head 512 to a desired position, the compressible material 210 may press against a body of the patient, thereby blocking the snap ring 230 or other portions of the scan tray 200 and the scan head 512 from touching the patient. As such, patient comfort may be enhanced.

Upon conclusion of the imaging, the hooks 240 may be unlocked, as illustrated in the example of FIG. 5 and the membrane may be removed. Due to the hydrophilicity of the membrane, ultrasound fluids may not contact other portions of the scan tray 200, thereby decreasing an operator clean-up time between patients.

In the example of FIG. 5, the hooks 240 may be integrated into sides of the scan head and allow the membrane 202, which may comprise chiffon and/or civflex, to be hooked onto the scan head rather than being fixed to the scan tray as in previous examples. Arranging the hooks 240 along more than one side of the scan head and/or scan tray may allow sufficient support in a center of the membrane to limit bunching of the membrane, which may affect an image quality.

In this way, a scan tray comprises a base, a snap ring, and a compressible material formed as one piece. The scan tray may reduce the demand for scan gel and/or scan lotion while also providing an acoustic membrane that is impenetrable and blocks fluids, such as the lotion or gel, from entering the scan head or contacting a transducer. The technical effect of the scan tray is to decrease cleaning times and the spread of contaminants from patient to patient.

FIGS. 1-5 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

An example of a system, comprises a scan tray comprising a snap ring arranged between a base and a compressible material, wherein the snap ring comprises at least one hook configured to releasably retain a membrane across the compressible material and onto the base.

A first example of the system further comprises an opening shaped via each of the base and the compressible material, the membrane extending over an entirety of the opening.

A second example of the system optionally including the first example, further comprises where the at least one hook is one of a plurality of hooks, where the plurality of hooks is arranged symmetrically along sides of the snap ring.

A third example of the system, optionally including one or more of the previous examples further includes where the compressible material is foam.

A fourth example of the system, optionally including one or more of the previous examples further includes where the compressible material contacts a patient during an ultrasound imaging procedure and blocks the snap ring, the at least one hook, and the base from contacting the patient.

A fifth example of the system, optionally including one or more of the previous examples further includes where the base is configured to mate with a scan head of an ultrasound device.

A sixth example of the system, optionally including one or more of the previous examples further includes where the ultrasound device is an automated breast ultrasound.

A seventh example of the system, optionally including one or more of the previous examples further includes where the membrane is a hydrophilic membrane.

An eighth example of the system, optionally including one or more of the previous examples further includes where the membrane is one or more of chiffon and civflex.

A ninth example of the system, optionally including one or more of the previous examples further includes where a scan head is coupled to the base and where the membrane does not contact the scan head while the membrane is coupled to the compressible material and the at least one hook.

A tenth example of the system, optionally including one or more of the previous examples further includes where the base is free of internal ribs.

An example of an ultrasound device, comprises a scan head comprising a transducer, a scan tray comprising a base, a snap ring, and a compressible material, wherein the base is configured to physically couple to the scan head, and a membrane configured to extend over the compressible material, wherein the snap ring comprises a plurality of hooks configured to releasably retain the membrane to the base and over the compressible material.

A first example of the ultrasound device further includes where the compressible material is closest to a patient during an ultrasound imaging procedure relative to the snap ring and the base, and wherein the membrane blocks ultrasound fluids from contacting one or more of the scan tray, the scan head, and the transducer.

A second example of the ultrasound device, optionally including the first example, further includes where the plurality of hooks comprises at least three hooks, and wherein each hook of the plurality of hooks is configured to engage with a recess shaped into the base, and wherein a portion of the membrane is pressed into the recess.

A third example of the ultrasound device, optionally including one or more of the previous examples, further includes where each hook of the plurality of hooks comprises a bend configured to articulate within a range to move to and away from the base.

A fourth example of the ultrasound device, optionally including one or more of the previous examples, further includes where each hook of the plurality of hooks extends from an outer body of the snap ring, and wherein the snap ring comprises plastic, silicon, or a combination thereof.

A fifth example of the ultrasound device, optionally including one or more of the previous examples, further includes where the snap ring is configured to sandwich the membrane between the compressible material and the base, wherein the membrane blocks the snap ring from directly contacting the base and the compressible material.

An example of a system, comprises a scan head for an automated breast ultrasound comprising a transducer, a scan tray configured to physically couple to the scan head via a base, the scan tray further comprising a snap ring and a compressible material, wherein the snap ring is arranged between the base and the compressible material, and an acoustic membrane configured to extend over an outside of the compressible material, wherein the acoustic membrane engages with a plurality of hooks of the snap ring and is retained into a plurality of recesses of the base via the plurality of hooks.

A first example of the system further includes where the base and the compressible material form a border extending around a continuous uninterrupted expanse and where the membrane extends across the expanse.

A second example of the system, optionally including the first example, further includes where the acoustic membrane is hydrophilic, and wherein the acoustic membrane is the only removable portion of the scan tray.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A system, comprising:
a scan tray comprising a snap ring arranged between a base and a compressible material, wherein the snap ring comprises at least one hook configured to releasably retain a membrane across the compressible material and onto the base.

2. The system of claim 1, further comprising an opening shaped via each of the base and the compressible material, the membrane extending over an entirety of the opening.

3. The system of claim 1, wherein the at least one hook is one of a plurality of hooks, where the plurality of hooks is arranged symmetrically along sides of the snap ring.

4. The system of claim 1, wherein the compressible material is foam.

5. The system of claim 1, wherein the compressible material contacts a patient during an ultrasound imaging procedure and blocks the snap ring, the at least one hook, and the base from contacting the patient.

6. The system of claim 1, wherein the base is configured to mate with a scan head of an ultrasound device.

7. The system of claim 6, wherein the ultrasound device is an automated breast ultrasound.

8. The system of claim 1, wherein the membrane is a hydrophilic membrane.

9. The system of claim 1, wherein the membrane is one or more of chiffon and civflex.

10. The system of claim 1, wherein a scan head is coupled to the base and where the membrane does not contact the scan head while the membrane is coupled to the compressible material and the at least one hook.

11. The system of claim 1, wherein the base is free of internal ribs.

12. An ultrasound device, comprising:
a scan head comprising a transducer;
a scan tray comprising a base, a snap ring, and a compressible material, wherein the base is configured to physically couple to the scan head; and
a membrane configured to extend over the compressible material, wherein the snap ring comprises a plurality of hooks configured to releasably retain the membrane to the base and over the compressible material.

13. The ultrasound device of claim 12, wherein the compressible material is closest to a patient during an ultrasound imaging procedure relative to the snap ring and the base, and wherein the membrane blocks ultrasound fluids from contacting one or more of the scan tray, the scan head, and the transducer.

14. The ultrasound device of claim 12, wherein the plurality of hooks comprises at least three hooks, and wherein each hook of the plurality of hooks is configured to engage with a recess shaped into the base, and wherein a portion of the membrane is pressed into the recess.

15. The ultrasound device of claim 12, wherein each hook of the plurality of hooks comprises a bend configured to articulate within a range to move to and away from the base.

16. The ultrasound device of claim 12, wherein each hook of the plurality of hooks extends from an outer body of the snap ring, and wherein the snap ring comprises plastic, silicon, or a combination thereof.

17. The ultrasound device of claim 12, wherein the snap ring is configured to sandwich the membrane between the compressible material and the base, wherein the membrane blocks the snap ring from directly contacting the base and the compressible material.

18. A system, comprising:
a scan head for an automated breast ultrasound comprising a transducer;
a scan tray configured to physically couple to the scan head via a base, the scan tray further comprising a snap ring and a compressible material, wherein the snap ring is arranged between the base and the compressible material; and
an acoustic membrane configured to extend over an outside of the compressible material, wherein the acoustic membrane engages with a plurality of hooks of the snap ring and is retained into a plurality of recesses of the base via the plurality of hooks.

19. The system of claim 18, wherein the base and the compressible material form a border extending around a continuous uninterrupted expanse and where the membrane extends across the expanse.

20. The system of claim 18, wherein the acoustic membrane is hydrophilic, and wherein the acoustic membrane is the only removable portion of the scan tray.
